(19) United States
(12) Patent Application Publication
(10) Pub. No.: US 2005/0100539 A1
(43) Pub. Date: May 12, 2005

(54) Enzymes useful for treating and methods for treating MPS-VI and cells lines for producing such enzymes recombinantly

(76) Inventor: Christopher M. Starr, Sonoma, CA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/658,699

(22) Filed: Sep. 9, 2003

Related U.S. Application Data
(62) Division of application No. 09/562,427, filed on May 1, 2000, now abandoned.

(51) Int. Cl. A61K 38/47

(52) U.S. Cl. 424/94.61

(57) ABSTRACT

The present invention provides a recombinant human N-acetylgalactosamine-4-sulfatase (rhASB) and biologically active mutants, fragments and analogs thereof as well as pharmaceutical formulations comprising recombinant human N-acetylgalactosamine-4-sulfatase (rhASB). The invention also provides methods for treating diseases caused all or in part by deficiencies in human N-acetylgalactosamine-4-sulfatase including MPS VI and methods for producing and purifying the recombinant enzyme.

### FIELD OF THE INTENTION

The present invention is in the field of clinical medicine, biochemistry and molecular biology. The present invention features therapeutics and methods for treating mucopolysaccharidosis VI as well as production and purification procedures for producing such therapeutics.

### BACKGROUND OF THE INVENTION

MPS VI (Maroteaux-Lamy syndrome) is a lysosomal storage disease in which the affected patients lack the enzyme N-acetylgalactosamine-4-sulfatase (ASB). The enzyme metabolizes the sulfate moiety of glycosaminoglycan (GAG) dermatan sulfate (Neufeld, et al., “The mucopolysaccharidoses” The Metabolic Basis of Inherited Disease, eds. Scriver et al., New York:McGraw-Hill, 1989, p. 1565-1587). In the absence of the enzyme, the stepwise degradation of dermatan sulfate is blocked and the substrate accumulates intracellulary in the lysosome in a wide range of tissues. The accumulation causes a progressive disorder with multiple organ and tissue involvement in which the infant appears normal at birth, but usually dies before puberty. The diagnosis of MPS VI is usually made at 6-24 months of age when children show progressive deceleration of growth, enlarged liver and spleen, skeletal deformities, coarse facial features, upper airway obstruction, and joint deformities. Progressive clouding of the cornea, communicating hydrocephalus, or heart disease may develop in MPS VI children. Death usually results from respiratory infection or cardiac disease. Distinct from MPS I, MPS VI is not typically associated with progressive impairment of mental status, although physical limitations may impact learning and development. Although most MPS VI patients have the severe form of the disease that is usually fatal by the teenage years, affected patients with a less severe form of the disease have been described which may survive for decades.

Several publications provide estimates of MPS VI incidence. A 1990 British Columbia survey of all births between 1952 and 1986 published by Lowry et al (Lowry, et al., Human Genet 85:389-390 (1990)) estimates an incidence of just 1:1,300,000. An Australian survey (Meikle et al., JAMA 281(3):249-54) of births between 1980-1996 found 18 patients for an incidence of 1:248,000. A survey in Northern Ireland (Nelson et al., Hum Genet 101:355-358 (1997)) estimated an incidence of 1:840,000. Finally, a survey from The Netherlands from 1970-1996 calculated a birth prevalence of 0.24 per 100,000 (Poorthuis et al., Hum. Genet 105:151-156 (1999)). Based on these surveys, it is estimated that there are between 50 and 300 patients in the U.S. who are diagnosed with all forms of this syndrome.

There is no satisfactory treatment for MPS VI although a few patients have benefited from bone marrow transplantation (BMT) (Krivit et al., N Engl J Med 311(25):1606-11 (1984)). (Krivit et al., Int. Pediatr 7:47-52 (1992)). BMT is not universally available for lack of a suitable donor and is associated with substantial morbidity and mortality. The European Group for Bone Marrow Transplantation reported transplant-related mortality of 10% (HLA identical) to 20-25% (HLA mismatched) for 63 transplantation cases of lysosomal disorders (Hoogerbrugge et al., Lancet 345: 1398-1402 (1995)). Other than BMT, most patients receive symptomatic treatment for specific problems as their only form of care. It is an object of the present invention to provide enzyme replacement therapy with recombinant human N-acetylgalactosamine-4-sulfatase (rhASB). No attempts to treat humans with rhASB have been made. Likewise, no acceptable clinical dosages or medical formulations have been provided. Several enzyme replacement trials in the feline MPS VI model have been conducted.

### SUMMARY OF THE INVENTION

In a first aspect, the present invention features novel methods of treating diseases caused all or in part by a deficiency in N-acetylgalactosamine-4-sulfatase (ASB). In one embodiment, this method features administering a recombinant N-acetylgalactosamine-4-sulfatase (ASB) or a biologically active fragment, mutant or analog thereof alone or in combination with a pharmaceutically suitable carrier. In other embodiments, this method features transferring a nucleic acid encoding all or a part of an N-acetylgalactosamine-4-sulfatase (ASB) or a biologically active mutant or analog thereof into one or more host cells in vivo. Preferred embodiments include optimizing the dosage to the needs of the organism to be treated, preferably mammals or humans, to effectively ameliorate the disease symptoms. In preferred embodiments the disease is mucopolysaccharidosis VI (MPS VI), Maroteaux-Lamy syndrome.

In a second aspect, the present invention features novel pharmaceutical compositions comprising an N-acetylgalactosamine-4-sulfatase (ASB) or a biologically active fragment, mutant or analog thereof useful for treating a disease caused all or in part by a deficiency in N-acetylgalactosamine-4-sulfatase (ASB). Such compositions may be suitable for administration in a number of ways such as parenteral, topical, intranasal, inhalation or oral administration. Within the scope of this aspect are embodiments featuring nucleic acid sequences encoding all or a part of an N-acetylgalactosamine-4-sulfatase (ASB) which may be administered in vivo into cells affected with an N-acetylgalactosamine-4-sulfatase (ASB) deficiency.

In a third aspect, the present invention features a method to produce an N-acetylgalactosamine-4-sulfatase (ASB) or a biologically active fragment, mutant or analog thereof in amounts which enable using the enzyme therapeutically. In a broad embodiment, the method comprises the step of transfecting a cDNA encoding for all or a part of a N-acetylgalactosamine-4-sulfatase (ASB) or a biologically active mutant or analog thereof into a cell suitable for the expression thereof. In some embodiments, a cDNA encoding for a complete N-acetylgalactosamine-4-sulfatase (ASB) is used, preferably a human N-acetylgalactosamine-4-sulfatase (ASB). However, in other embodiments, a cDNA encoding for a biologically active fragment or mutant thereof may be used. Specifically, one or more amino acid substitutions may be made while preserving or enhancing the biological activity of the enzyme. In other preferred embodiments, an expression vector is used to transfer the cDNA into a suitable cell or cell line for expression thereof. In one particularly preferred embodiment, the cDNA is transfected into a Chinese hamster ovary cell to create cell line CHO K1. In yet other preferred embodiments, the production procedure comprises the following steps: (a) growing cells transfected with a DNA encoding all or a biologically active fragment or mutant of a human N-acetylgalactosamine-4-sulfatase in a suitable growth medium to an appropriate density, (b) introducing the transfected cells into a bioreactor, (c) supplying a suitable growth medium to the bioreactor, and (d) separating the transfected cells from the media containing the enzyme.

In a fourth aspect, the present invention provides a transfected cell line which features the ability to produce N-acetylgalactosamine-4-sulfatase (ASB) in amounts which enable using the enzyme therapeutically. In preferred embodiments, the present invention features a recombinant Chinese hamster ovary cell line such as the CHO K1 cell line that stably and reliably produces amounts of an N-acetylgalactosamine-4-sulfatase (ASB) or a biologically active fragment, mutant or analog thereof which enable using the enzyme therapeutically. Especially preferred is the CHO-K1 cell line designated CSL4S-342. In some preferred embodiments, the cell line may contain at least about 10 copies of an expression construct. In even more preferred embodiments, the cell line expresses the recombinant N-acetylgalactosamine-4-sulfatase (ASB) or a biologically active fragment, mutant or analog thereof in amounts of at least about 20-40 micrograms per 10⁷ cells per day.

In a fifth aspect, the present invention provides novel vectors suitable to produce N-acetylgalactosamine-4-sulfatase (ASB) or a biologically active fragment, mutant or analog thereof in amounts which enable using the enzyme therapeutically.

In a sixth aspect, the present invention provides novel N-acetylgalactosamine-4-sulfatase (ASB) or a biologically active fragment, mutant or analog thereof produced in accordance with the methods of the present invention and thereby present in amounts which enable using the enzyme therapeutically. The specific activity of the N-acetylgalactosamine-4-sulfatase (ASB) according to the present invention is preferably in the range of 20-90 units, and more preferably greater than about 50 units per mg protein.

In a seventh aspect, the present invention features a novel method to purify N-acetylgalactosamine-4-sulfatase (ASB) or a biologically active fragment, mutant or analog thereof. According to a first embodiment, a transfected cell mass is grown and removed leaving recombinant enzyme. Exogenous materials should normally be separated from the crude bulk to prevent fouling of the columns. Preferably, the growth medium containing the recombinant enzyme is passed through an ultrafiltration and diafiltration step. In another preferred embodiment, the filtered solution is passed through a DEAE Sepharose chromatography column, then a Blue Sepharose chromatography column, then a Cu++ Chelating Sepharose chromatography column, and then a Phenyl Sepharose chromatography column. Such a four step column chromatography including using a DEAE Sepharose, a Blue Sepharose, a Cu++ Chelating Sepharose and a Phenyl Sepharose chromatography column sequentially results in especially highly purified recombinant enzyme. Those skilled in the art readily appreciate that one or more of the chromatography steps may be omitted or substituted, or that the order of the chromatography steps may be changed within the scope of the present invention. In other preferred embodiments, the eluent from the final chromatography column is ultrafiltered/diafiltered, and an appropriate step is performed to remove any remaining viruses. Finally, appropriate sterilizing steps may be performed as desired.

### DESCRIPTION OF THE FIGURES

FIG. 1 provides a flow diagram of the method for producing a human N-acetylgalactosamine-4-sulfatase (ASB) according to the present invention.

FIG. 2 provides a flow diagram of the method for purifying a human N-acetylgalactosamine-4-sulfatase (ASB) according to the present invention.

FIG. 3 shows the purity analysis of rhASB by SDS PAGE and Western blotting.

FIG. 4 shows the purity analysis after each chromatography purification.

### DETAILED DESCRIPTION OF THE INVENTION

In a first aspect, the present invention features novel methods of treating diseases caused all or in part by a deficiency in N-acetylgalactosamine-4-sulfatase (ASB). In one embodiment, this method features administering a recombinant N-acetylgalactosamine-4-sulfatase (ASB) or a biologically active fragment, mutant or analog thereof alone or in combination with a pharmaceutically suitable carrier. In other embodiments, this method features transferring a nucleic acid encoding, all or a part of an N-acetylgalactosamine-4-sulfatase (ASB) or a biologically active mutant thereof into one or more host cells in vivo. Preferred embodiments include optimizing the dosage to the needs of the organism to be treated, preferably mammals or humans, to effectively ameliorate the disease symptoms. In preferred embodiments the disease is mucopolysaccharidosis VI (MPS VI), Maroteaux-Lamy syndrome.

The indication for recombinant human N-acetylgalactosamine-4-sulfatase (rhASB) is for the treatment of MPS VI, also known as Maroteaux-Lamy Syndrome. According to preferred embodiments, an initial dose of 1 mg/kg (˜50 U/kg) is provided to patients suffering from a deficiency in N-acetylgalactosamine-4-sulfatase. Preferably, the N-acetylgalactosamine-4-sulfatase is administered weekly by injection. According to other preferred embodiments, patients who do not demonstrate a reduction in urinary glycosaminoglycan excretions of at least fifty percent are changed to a dosage of 2 mg/kg (˜100 U/kg) within about three months of initial dosage. Preferably, the N-acetylgalactosamine-4-sulfatase (rhASB) or a biologically active fragment, mutant or analog thereof is administered intravenously over approximately a four-hour period once weekly preferably for as long as significant clinical symptoms of disease persist. Also, preferably, the N-acetylgalactosamine-4-sulfatase (rhASB) is administered by an intravenous catheter placed in the cephalic or other appropriate vein with an infusion of saline begun at about 30 cc/hr. Further, preferably the N-acetylgalactosamine-4-sulfatase (rhASB) is diluted into about 100 cc of normal saline supplemented with about 1 mg/ml human albumin.

In a second aspect, the present invention features novel pharmaceutical compositions comprising human N-acetylgalactosamine-4-sulfatase (rhASB) or a biologically active fragment, mutant or analog thereof useful for treating a deficiency in N-acetylgalactosamine-4-sulfatase. The recombinant enzyme may be administered in a number of ways in addition to the preferred embodiments described above, such as parenteral, topical, intranasal, inhalation or oral administration. Another aspect of the invention is to provide for the administration of the enzyme by formulating it with a pharmaceutically-acceptable carrier which may be solid, semi-solid or liquid or an ingestable capsule. Examples of pharmaceutical compositions include tablets, drops such as nasal drops, compositions for topical application such as ointments, jellies, creams and suspensions, aerosol inhalation, nasal spray, liposomes. Usually the recombinant enzyme comprises between 0.05 and 99% or between 0.5 and 99% by weight of the composition, for example between 0.5 and 20% for compositions intended for injection and between 0.1 and 50% for compositions intended for oral administration.

To produce pharmaceutical compositions in this form of dosage units for oral application containing a therapeutic enzyme, the enzyme may be mixed with a solid, pulverulent carrier, for example lactose, saccharose, sorbitol, mannitol, a starch such as potato starch, corn starch, amylopectin, laminaria powder or citrus pulp powder, a cellulose derivative or gelatine and also may include lubricants such as magnesium or calcium stearate or a Carbowax or other polyethylene glycol waxes and compressed to form tablets or cores for dragees. If dragees are required, the cores may be coated for example with concentrated sugar solutions which may contain gum arabic, talc and/or titanium dioxide, or alternatively with a film forming agent dissolved in easily volatile organic solvents or mixtures of organic solvents. Dyestuffs can be added to these coatings, for example, to distinguish between different contents of active substance. For the composition of soft gelatine capsules consisting of gelatine and, for example, glycerol as a plasticizer, or similar closed capsules, the active substance may be admixed with a Carbowax or a suitable oil as e.g., sesame oil, olive oil, or arachis oil. Hard gelatine capsules may contain granulates of the active substance with solid, pulverulent carriers such as lactose, saccharose, sorbitol, mannitol, starches such as potato starch, corn starch or amylopectin, cellulose derivatives or gelatine, and may also include magnesium stearate or stearic acid as lubricants.

Therapeutic enzymes of the present invention may also be administered parenterally such as by subcutaneous, intramuscular or intravenous injection either by single injection or pump infusion or by sustained release subcutaneous implant, and therapeutic enzymes may be administered by inhalation. In subcutaneous, intramuscular and intravenous injection the therapeutic enzyme (the active ingredient) may be dissolved or dispersed in a liquid carrier vehicle. For parenteral administration the active material may be suitably admixed with an acceptable vehicle, preferably of the vegetable oil variety such as peanut oil, cottonseed oil and the like. Other parenteral vehicles such as organic compositions using solketal, glycerol, formal, and aqueous parenteral formulations may also be used.

For parenteral application by injection, compositions may comprise an aqueous solution of a water soluble pharmaceutically acceptable salt of the active acids according to the invention, desirably in a concentration of 0.5-10%, and optionally also a stabilizing agent and/or buffer substances in aqueous solution. Dosage units of the solution may advantageously be enclosed in ampoules.

When therapeutic enzymes are administered in the form of a subcutaneous implant, the compound is suspended or dissolved in a slowly dispersed material known to those skilled in the art, or administered in a device which slowly releases the active material through the use of a constant driving-force such as an osmotic pump. In such cases administration over an extended period of time is possible.

For topical application, the pharmaceutical compositions are suitably in the form of an ointment, cell, suspension, cream or the like. The amount of active substance may vary, for example between 0.05-20% by weight of the active substance. Such pharmaceutical compositions for topical application may be prepared in known manner by mixing, the active substance with known carrier materials such as isopropanol, glycerol, paraffin, stearyl alcohol, polyethylene glycol, etc. The pharmaceutically acceptable carrier may also include a known chemical absorption promoter. Examples of absorption promoters are, e.g., dimethylacetamide (U.S. Pat. No. 3,472,931), trichloro ethanol or trifluoroethanol (U.S. Pat. No. 3,891,757), certain alcohols and mixtures thereof (British Patent No. 1,001,949). A carrier material for topical application to unbroken skin is also described in the British patent specification No. 1,464,975, which discloses a carrier material consisting of a solvent comprising 40-70% (v/v) isopropanol and 0-60% (v/v) glycerol, the balance, if any, being an inert constituent of a diluent not exceeding 40% of the total volume of solvent.

The dosage at which the therapeutic enzyme containing pharmaceutical compositions are administered may vary within a wide range and will depend on various factors such as for example the severity of the disease, the age of the patient, etc., and may have to be individually adjusted. As a possible range for the amount of therapeutic enzyme which may be administered per day be mentioned from about 0.1 mg—to about 2000 mg or from about 1 mg to about 2000 mg.

The pharmaceutical compositions containing the therapeutic enzyme may suitably be formulated so that they provide doses within these ranges either as single dosage units or as multiple dosage units. In addition to containing a therapeutic enzyme (or therapeutic enzymes), the subject formulations may contain one or more substrates or cofactors for the reaction catalyzed by the therapeutic enzyme in the compositions. Therapeutic enzyme containing, compositions may also contain more than one therapeutic enzyme. Likewise, the therapeutic enzyme may be in conjugate form being bound to another moiety, for instance PEG. Additionally, the therapeutic enzyme may contain one or more targeting moieties or transit peptides to assist delivery to a tissue, organ or organelle of interest.

The recombinant enzyme employed in the subject methods and compositions may also be administered by means of transforming patient cells with nucleic acids encoding the N-acetylgalactosamine-4-sulfatase or a biologically active fragment, mutant or analog thereof. The nucleic acid sequence so encoding may be incorporated into a vector for transformation into cells of the patient to be treated. Preferred embodiments of such vectors are described herein. The vector may be designed so as to integrate into the chromosomes of the subject, e.g., retroviral vectors, or to replicate autonomously in the host cells. Vectors containing encoding N-acetylgalactosamine-4-sulfatase nucleotide sequences may be designed so as to provide for continuous or regulated expression of the enzyme. Additionally, the genetic vector encoding the enzyme may be designed so as to stably integrate into the cell genome or to only be present transiently. The general methodology of conventional genetic therapy may be applied to polynucleotide sequences encoding- N-acetylgalactosamine-4-sulfatase. Reviews of conventional genetic therapy techniques can be found in Friedman. Science 244:1275-1281 (1989); Ledley, J Inherit. Aletab. Dis. 13:587-616(1990); and Tososhev et al., Curr Opinions Biotech. 1:55-61 (1990).

A particularly preferred method of administering the recombinant enzyme is intravenously. A particularly preferred composition comprises recombinant N-acetylgalactosamine-4-sulfatase, normal saline, phosphate buffer to maintain the pH at about 5-7, and human albumin. The composition may additionally include polyoxyethylenesorbitan 20 or 80 (Tween −20 or Tween −80) to improve the stability and prolong shelf life. These ingredients may be provided in the following amounts:

| | | |
|---|---|---|
| N-acetylgalactosamine- | 1-5 | mg/ml or 50-250 units/ml |
| 4-sulfatase (rhASB) | | |
| Sodium chloride solution | 150 | mM in an IV bag, 50-250 cc |
| | | total volume |
| Sodium phosphate buffer | 10-100 | mM, pH 5.8 |
| Human albumin | 1 | mg/mL |
| Tween-20 or Tween-80 | 0.001% | (w/v) |

In a third aspect, the present invention features a method to produce N-acetylgalactosamine-4-sulfatase (ASB) or a biologically active fragment, mutant or analog thereof in amounts which enable using the enzyme therapeutically. In a broad embodiment, the method comprises the step of transfecting a cDNA encoding for all or a part of a N-acetylgalactosamine-4-sulfatase (ASB) or a biologically active mutant or analog thereof into a cell suitable for the expression thereof. In some embodiments, a cDNA encoding for a complete N-acetylgalactosamine-4-sulfatase (ASB) is used, preferably a human N-acetylgalactosamine-4-sulfatase (ASB). However, in other embodiments, a cDNA encoding for a biologically active fragment or mutant thereof may be used. Specifically, one or more amino acid substitutions may be made while preserving or enhancing the biological activity of the enzyme. In other preferred embodiments, an expression vector is used to transfer the cDNA into a suitable cell or cell line for expression thereof. In one Particularly preferred embodiment, the cDNA is transfected into a Chinese hamster ovary cell to create cell line CHO K1. In yet other preferred embodiments, the production procedure comprises the following steps: (a) growing cells transfected with a DNA encoding all or a biologically active fragment or mutant of a human N-acetylgalactosamine-4-sulfatase a suitable growth medium to an appropriate density, (b) introducing the transfected cells into a bioreactor, (c) supplying a suitable growth medium to the bioreactor, (d) harvesting said medium containing the recombinant enzyme, and (e) substantially removing the transfected cells from the harvest medium. A preferred medium for growing the transfected cells is a JRH Excell 302 medium supplemented with L-glutamine, glucose and hypoxanthine/thymidine in addition to G418. It is preferred to grow the cells in such a medium to achieve a cell density of about 1×10⁷ resulting in 10-40 mg/ml of active enzyme. Moreover, it is preferable to grow the transfected cells in a bioreactor for about 5 to 15 days, most preferably about 9 days. According to preferred embodiments, the transfected cells may be substantially removed from the bioreactor supernatant by filtering them through successive membranes such as a 10 μm membrane followed by a 1 μm membrane followed by a 0.2 μm. Any remaining harvest medium may be discarded prior to filtration.

Recombinant human N-acetylgalactosamine-4-sulfatase may be produced in Chinese hamster ovary cells (Peters, et al. J. Biol. Chem. 265:3374-3381). Its uptake is mediated by a high affinity mannose-6-phosphate receptor expressed on most, if not all, cells Neufeld et al., “The mucopolysaccharidoses” The Metabolic Basis of Inherited Disease, eds. Scriver et al. New York:McGraw-Hill (1989) p. 1565-1587). Once bound to the mannose-6-phosphate receptor, the enzyme is endocytosed through coated pits and transported to the lysosomes. At the pH of lysosomes, the enzyme is active and begins removing sulfate residues from accumulated dermatan sulfate. In MPS VI fibroblasts, the clearance of storage is rapid and easily demonstrated within 92 hours of enzyme exposure (Anson et al. J.Clin.Invest. 99:651-662 (1997)).

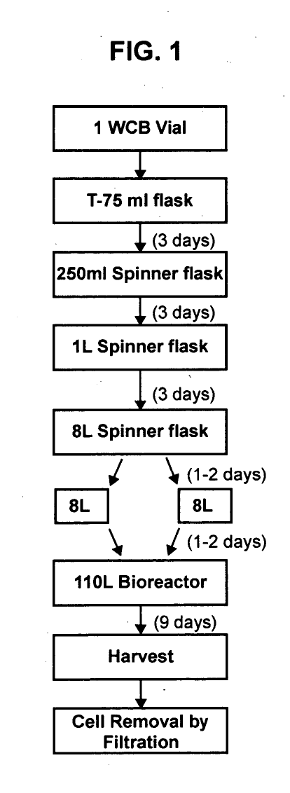

The recombinant enzyme may be produced at a 110-L (approximately 90 L working volume) fermentation scale according to a process according to the flow diagram outlined in FIG. 1.

A more detailed description of one preferred production process according to the methods of the present invention is set forth in Table 1.

TABLE 1

| Step | Process | In-Process Testing |
|---|---|---|
| 1. Thawing of the | Inoculate the thawed cells into one T-75 | Cell count |
| Working Cell Bank | flask with 25 mL of JRH Exell 302 medium | Cell viability |
| (WCB) | supplemented with 4 mM L-glutamine, 4.5 g/L | |
| | glucose and 10 mg/L | |
| | hypoxanthine/thymidine plus G418 | |
| | Culture for 3 days to achieve 1 × 10¹⁰ cell | |
| | density | |
| | ↓ | |
| 3. 250 mL Spinner | Add cells to 175 mL of supplemented | Cell count |
| Flask | medium plus G418 | Cell viability |
| | Culture for 3 days | |
| | ↓ | |
| 4. 1 L Spinner Flask | Add cells to 800 mL of supplemented | Cell count |
| | medium plus G418 | Cell viability |
| | Culture for 1-2 days | |
| | ↓ | |
| 5. 8 L Spinner Flask | Add cells to 4 L of supplemented medium | Cell count |
| | plus G418 | Cell viability |
| | Culture for 1-2 days | |
| | ↓ | |
| 6. 2 × 8 L Spinner | Split working volume into 2 8 L Spinner | Cell count |
| Flask | Flasks | Cell viability |
| | Add cells to 5.5 L of supplemented | |
| | medium plus G418 to each 8 L Spinner | |
| | Flask | |
| | Culture for 1-2 days | |
| | ↓ | |
| 7. Inoculation of 110 L | Add cells to 7 mL of supplemented | Cell count |
| Bioreactor | medium | Cell viability |
| | Culture 9 days | |
| 8. Production | Approximately 9 days of growth in | Cell Count |
| | bioreactor | Cell viability |
| | | Activity |
| | ↓ | |
| 9. Harvest | Harvest is pumped into 100 L bag, | |
| Supernatant | refrigerated overnight | |
| | ↓ | |
| 10. Cell Removal | Cells are removed from the harvest | QC Release Point |
| | medium by filtration through a 10 μm | Activity |
| | membrane cartridge followed by 1 μm and | Bioburden |
| | 0.2 μm cartridges. Since the cells have been | Endotoxin |
| | allowed to settle overnight the final 5 to | Mycoplasma |
| | 10% of the harvest medium is discarded | In vitro advent. Agents |
| | prior to filtration. | |

In a fourth aspect, the present invention provides a transfected cell line which features the ability to produce N-acetylgalactosamine-4-sulfatase (ASB) or a biologically active fragment, mutant or analog thereof in amounts which enable using the enzyme therapeutically. In preferred embodiments, the present invention features a recombinant Chinese hamster ovary cell line such as the CHO K1 cell line that stably and reliably produces amounts of N-acetylgalactosamine-4-sulfatase (ASB) which enable using the enzyme therapeutically. Especially preferred is the CHO-K1 cell line designated CSL4S-342. In some preferred embodiments, the cell line may contain at least about 10 copies of an expression construct. In even more preferred embodiments, the cell line expresses recombinant N-acetylgalactosamine-4-sulfatase (ASB) in amounts of at least about 40-80 micrograms per 10⁷ cells per day.

Recombinant human N-acetylgalactosamine-4-sulfatase (rhASB) may be produced from a stable transfected CHO-DK1 (Chinese hamster ovary) cell line designated CSL4S-342. The cell line is described in the literature (Crawley, J.Clin.Invest. 99:651-662 (1997)). Master Cell Bank (MCB) and Working Cell Bank (WBC) were prepared at Tektagen Inc. (Malvem, Pa.). The cell banks have been characterized per ICH recommended guidelines for a recombinant mammalian cell line.

In a fifth aspect, the present invention provides novel vectors suitable to produce N-acetylgalactosamine-4-sulfatase (ASB) or a biologically active fragrnent, mutant or analog thereof in amounts which enable using the enzyme therapeutically.

In a sixth aspect, the present invention provides novel N-acetylgalactosamine-4-sulfatase (ASB) or a biologically active fragment, mutant or analog thereof produced in accordance with the methods of the present invention and thereby present in amounts which enable using the enzyme therapeutically. The preferred specific activity of the N-acetylgalactosamine-4-sulfatase (ASB) according to the present invention is about 20-90 Unit, and more preferably greater than 50 units per milligram protein. Preferably, the enzyme has a deglycosylated weight of about 55 to 56 kDa, most preferably about 55.7 kDa. Preferably, the enzyme has a glycosylated weight of about 63 to 65 kDa, most preferably about 64 kDa. The present invention also includes biologically active fragments including truncated molecules, analogs and mutants of the naturally-occurring human N-acetylgalactosamine-4-sulfatase.

The human cDNA for N-acetylgalactosamine-4-sulfatase predicts a protein of 533 amino acids with a signal peptide of 41 amino acids (Peters, et al. J. Biol. Chem. 265:3374-3381). The predicted molecular weight is 55.7 kDa after signal peptide cleavage. The recombinant enzyme has an apparent molecular weight of 64 kDa on SDS-PAGE due to carbohydrate modifications. The predicted protein sequence contains six potential N-linked oligosaccharide modification sites of which four may be used based on a 2,000 kDa average mass and 8,000 kDa difference between predicted and apparent mass. A mature form of the intracellular protein has three peptides attached by cystine bonds. The largest peptide has a molecular weight of 47 kDa; the other two has a molecular weight of 6 and 7 kDa respectively.

A description of a drug product produced and purified according to the methods of the present invention is provided in Table 2.

TABLE 2
Drug Product Preliminary Specifications

| Test | Procedure | Specification |
|---|---|---|
| Activity | Fluorescence assay | 20,000-120,000 mUnits |
| Adventitious Viruses* | In Vitro Assay | Pass |
| Appearance | Visual | Clear, colorless to pale |
| | | yellow solution |
| Bacterial Endotoxin | LAL | ≦2 EU/mL |
| Chloride | Atomic Absorption | Report Value |
| ASB fibroblast Uptake Assay | TBD | ≦40 nmol |
| Mycoplasma* | Points to Consider 1993 | Pass |
| Particulates | USP | ≦600/vial at 25 μm & |
| | | ≦6000/vial at 10 μm |
| PH | USP | 5.5-6.8 |
| Phosphate | Atomic Absorption | Report Value |
| Protein Concentration | UV 280 | 0.8-1.2 mg/ml |
| Purity | SDS PAGE | 1 major brand between 65-70 kDa |
| | RP-HPLC | >95% |
| Residual Blue Dye | TBD | Report Value |
| Residual Copper | TBD | Report Value |
| Sodium | Atomic Absorption | Report Value |
| Specific Activity | Calculation | 40,000-80,000 mUnits/mg |
| Sterility | 21 CFR 610 | Pass |

*Tested on harvested supernatant from bioreactor (after cell removal by filtration).

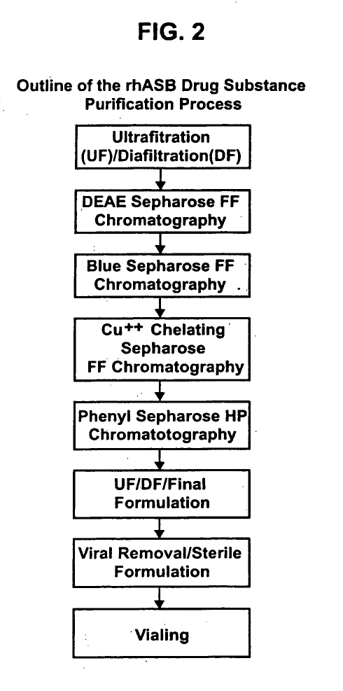

In a seventh aspect, the present invention features a novel method to purify N-acetylgalactosamine-4-sulfatase (ASB) or a biologically active fragment, mutant or analog thereof. According to a first embodiment, a transfected cell mass is grown and removed leaving recombinant enzyme. Exogenous materials should normally be separated from the crude bulk to prevent fouling of the columns. Preferably, the growth medium containing the recombinant enzyme is passed through an ultrafiltration and diafiltration step. In another preferred embodiment, the filtered solution is passed through a DEAE Sepharose chromatography column, then a Blue Sepharose chromatography column, then a Cu++ 20 Chelating Sepharose chromatography column, and then a Phenyl Sepharose chromatography column. Such a four step column chromatography including using a DEAE Sepharose, a Blue Sepharose, a Cu++ Chelating Sepharose and a Phenyl Sepharose chromatography column sequentially results in especially highly purified recombinant enzyme. Those of skill in the art appreciate that one or more chromatography steps may be omitted or substituted or the order of the steps altered within the scope of the present invention. In other preferred embodiments, the eluent from the final chromatography column is ultrafiltered/diafiltered, and an appropriate step is performed to remove any remaining viruses. Finally, appropriate sterilizing steps may be performed as desired. The recombinant enzyme may be purified according to a process outlined in FIG. 2. The quality of the recombinant enzyme is key to patients. As shown in FIGS. 3 and 4, rhASB produced by the present invention is substantially (>95%) pure.

In preferred embodiments, the ultrafiltration/diafiltration step is performed with a sodium phosphate solution of about 10 mM and with a sodium chloride solution of about 100 mM at a pH of about 7.3. In further preferred embodiments, the DEAE Sepharose chromatography step is performed at a pH of about 7.3 wherein the elute solution is adjusted with an appropriate buffer, preferably a sodium chloride and sodium phosphate buffer. In additional preferred embodiments, the Blue Sepharose chromatography step is performed at a pH of about 5.5 wherein the elute solution is adjusted with an appropriate buffer, preferably a sodium chloride and sodium acetate buffer. Also, in preferred embodiments, the Cu++ Chelating Sepharose chromatography step is performed with an elution buffer including sodium chloride and sodium acetate. In especially preferred embodiments, a second ultrafiltration/diafiltration step is performed on the eluate from the chromatography runs wherein the recombinant enzyme is concentrated to a concentration of about 1 mg/ml in a formulation buffer such as a sodium chloride and sodium phosphate buffer to a pH of about 5.5 to 6.0, most preferably to a pH of 5.8. Phosphate buffer is a preferred buffer used in the process because phosphate buffer prevents critical degradation and improves the stability of the enzyme.

A more detailed description of particularly preferred purification methods within the scope of the present invention is set forth in Table 3.

TABLE 3
Purification Process Overview

| Step | Process | |
|---|---|---|
| 1. UF/DF | Filtered harvest fluid (HF) is concentrated ten fold and then diafiltered with | |
| | 5 volumes of 10 mM Sodium Phosphate, 100 mM NaCl, pH 7.3 using a | |
| | tangential flow filtration (TFF) system. | |
| | ↓ | |
| 2. DEAE | Pre-wash 1 buffer: | 0.1 N NaOH |
| Sepharoe FF | Pre-wash 2 buffer: | 100 mM NaPO4 pH 7.3 |
| (flow through) | Equilibration buffer: | 100 mM NaCl, 10 mM NaPO4, pH 7.3 |
| | Load: | Product from Step 1 |
| | Wash buffer: | 100 mM NaCl, 10 mM NaPO4, pH 7.3 |
| | Strip buffer: | 1 M NaCl, 10 mM NaPO4, pH 7.3 |
| | Sanitization buffer: | 0.5 N NaOH |
| | Storage buffer: | 0.1 N NaOH |
| | ↓ | |
| 3. Blue Sepharoe | Pre-wash 1: | 0.1 N NaOH |
| FF | Pre-wash 2: | H₂O |
| | Pre-wash 3: | 0.5 M NaAc, pH 5.5 |
| | Equilibration buffer: | 150 mM NaCl, 20 mM NaAc, pH 5.5 |
| | Load: | DEAE flow through |
| | Wash buffer: | 150 mM NaCl, 20 mM NaAc, pH 5.5 |
| | Elution buffer: | 500 mM NaCl, 20 mM NaAc, pH 5.5 |
| | Regeneration buffer: | 1 M NaCl, 20 mM NaAc, pH 5.5 |
| | Sanitization buffer: | 0.1 N NaOH, 0.5-2 hours |
| | Storage buffer: | 500 mM NaCl, 20 mM NaAc, pH 5.5, |
| | | 20% ETOH |
| | ↓ | |
| 4. Cu++ | Sanitization buffer: | 0.1 N NaOH |
| Chelating | Wash buffer: | H₂O |
| Sepharoe FF | Charge Buffer: | 0.1 M Copper Sulfate |
| | Equilibration buffer: | 20 mM NaAc, 0.5 M NaCl, 10% |
| | | Glycerol, pH 6.0 |
| | Load: | Blue Sepharose Eluate |
| | Wash Buffer 1: | 20 mM NaAc, 0.5 M NaCl, 10% |
| | | Glycerol, pH 6.0 |
| | Wash Buffer 2: | 20 mM NaAc, 1 M NaCl, 10% Glycerol, |
| | | PH 4.0 |
| | Wash Buffer 3: | 20 mM NaAc, 1 M NaCl, 10% Glycerol, |
| | | PH 3.8 |
| | Elution Buffer: | 20 mM NaAc, 1 M NaCl, 10% Glycerol, |
| | | PH 3.6 |
| | Strip Buffer: | 50 mM EDTA, 1 M NaCl |
| | Sanitization Buffer: | 0.5 N NaOH, 0.5-2 hours |
| | Storage Buffer: | 0.1 N NaOH |
| | ↓ | |
| 5. Phenyl | Pre-wash 1 Buffer: | 0.1 N NaOH |
| Sepharoe HP | Pre-wash 2 Buffer: | H₂O |
| | Equilibration buffer: | 3 M NaCl, 20 mM NaAc, pH 4.5 |
| | Load: | Cu⁺⁺ Chelating Sepharose Flow through |
| | Wash Buffer 1: | 1.5 M NaCl, 20 mM NaAc, pH 4.5 |
| | Wash Buffer 2: | 1.5 M NaCl, 20 mM NaAc, pH 4.5 |
| | Elution buffer 1: | 1.0 M NaCl, 20 mM, NaAc, pH 4.5 |
| | Strip Buffer: | 0 M NaCl, 20 mM NaAc, pH 4.5 |
| | Sanitization Buffer: | 0.5 N NaOH |
| | Storage Buffer: | 0.1 N NaOH |
| | ↓ | |

| | |
|---|---|
| 6. UF/DF | The purified rhASB is concentrated and diafiltered to a final concentration of |
| | 1 mg/ml in formulation buffer (150 mM NaCl, 10 mM NaPO4, pH 5.8) |
| | using a TFF system. |
| | ↓ |
| 7. Formulation | Dilute with additional formulation buffer to 1.0 mg/ml |
| (If necessary) | |
| | ↓ |
| 8. Viral | 0.04 μm filtration into sterile container |
| Reduction/ | |
| Sterile filtration | |
| | ↓ |
| 9. Vialing | Product filled into 5 cc Type 1 glass vials, manually stoppered, crimped and |
| | labeled. |

In especially preferred embodiments, the formulated bulk drug substance may be sterilized through a 0.04 micron filter in a class 100 laminar flow hood into Type 1 glass vials. The vials may be filled to a final volume of about 5 mL using a semi-automatic liquid filling machine. The vials may then be manually stoppered, sealed and labeled.

The components of the drug product thus obtained are set forth in Table 4. The components of the drug product composition within the scope of the present invention are set forth in Table 5.

TABLE 4
Drug Product Component

| Component | Description |
|---|---|
| Active Ingredient | Recombinant human N-acetylgalactosamine- |
| | 4-sulfatase |
| Excipients | Sodium Phosphate, Monobasic, 1 H₂0 |
| | Sodium Phosphate, Dibasic, 7 H₂0 |
| | Sodium Chloride |
| Container | Kimble Glass, Type I 5 ml clear glass vial, |
| | Borosilitcate West pharmaceuticals, |
| | S-127 4432150 Grey stopper |

TABLE 5
Drug Product Composition

| Component | Amount |
|---|---|
| RhASB | 1 mg/mL |
| Sodium Phosphate, Monobasic, 1 H₂0 | 9 mM |
| Sodium Phosphate, Dibasic, 7 H₂0 | 1 mM |
| Sodium Chloride | 150 mM |

The invention having been described, the following- examples are offered to illustrate the subject invention by way of illustration, not by way of limitation.

### EXAMPLE 1

### Clinical Evaluation with Recombinant Human N-acetylgalactosamine-4-sulfatase (rhASB)

Summary

The indication for recombinant human N-acetylgalactosamine-4-sulfatase (rhASB) is the treatment of MPS VI, also known as Maroteaux-Lamy Syndrome. We propose a clinical development program for rhASB consisting of an initial open-label clinical trial that will provide an assessment of weekly infusions of the enzyme for safety, pharmacokinetics, and initial response of both surrogate and defined clinical endpoints. The trial will be conducted for a minimum of three months to collect sufficient safety information for 5 evaluable patients. At this time, should the initial dose of 1 mg/kg not produce a reasonable reduction in excess urinary glycosaminoglycans or produce a significant direct clinical benefit, the dose will be doubled and maintained for an additional three months to establish safety and to evaluate further efficacy.

Objectives

Our primary objective is to demonstrate safety of a weekly infusion of rhASB in patients with MPS VI for a minimum of a three-month period. Measurements of safety will include adverse events, immune response and allergic reactions (complement activation, antibody formation to recombinant enzyme), complete clinical chemistry panel (kidney and liver function), urinalysis, and CBC with differential.

One secondary objective is to evaluate efficacy by monitoring changes in several parameters known to be affected in MPS VI. These include a six-minute walk test (as a measure of exercise tolerance), full pulmonary function (PFT) evaluation, reduction in levels of urinary glycosaminoglycans and hepatomegaly (as measures of kidney and liver GAG storage), growth velocity, joint range of motion, Children's Health Assessment Questionnaire (CHAQ), visual acuity, cardiac function, sleeping studies, and two different global assessments; one performed by the investigator, one performed by the patient/caregiver. A second secondary objective is to determine pharmacokinetic parameters of infused drug in the circulation, and general distribution and half-life of intracellular enzyme using leukocytes and buccal tissue as sources of tissue. It is anticipated that these measures will help relate dose to clinical response based on the levels of enzyme delivered to the lysosomes of cells.

Methods

We will conduct a single center, open-labeled study to demonstrate safety and to evaluate clinical parameters of treatment with rhASB in patients with MPS VrI. Patients will be admitted for a two week baseline evaluation that will include a medical history and physical exam, psychological testing, endurance testing (treadmill), a standard set of clinical laboratory tests (CBC, Panel 20, CH50, UA), a MRI or CAT scan of the body (liver and spleen volumetric determination, bone and bone marrow evaluation, and lymph node and tonsillar size), a cardiology evaluation (echocardiogram, EKG, CXR), an airway evaluation (pulmonary function tests), a sleep study to evaluate for obstructive events during sleep, a joint restriction analysis (range of motion will be measured at the elbows and interphalangeal joints), a LP with CNS pressure, and biochemical studies (buccal N-acetylgalactosamine-4-sulfatase activity on two occasions, leukocyte N-acetylgalactosamine-4-sulfatase activity on two occasions, urinary GAG on three occasions, serum generation for ELISA of anti-rhASB antibodies and 24 hour urine for creatinine clearance). In addition to the above evaluations, each patient will be photographed and videotaped performing some physical movements such as attempting to raise their hands over their heads and walking. Patients will be titrated with antihistamines such that pretreatment with these agents could be effectively employed prior to infusion of enzyme. The proposed human dose of 1 mg/kg (50 U/kg) will be administered weekly by i.v. infusion over 4 hours. The patient will remain in the hospital for the first two weeks, followed by short stays for the next four weeks. Treatment for the final six weeks will be conducted at a facility close to the patient's home. Patients will return to the hospital for a complete evaluation at three months. Should dose escalation to 2 mg/kg be required, the patients will follow the same schedule outlined above for the first twelve weeks. Under either scenario, a complete evaluation will also occur at 6 months from the time of entering the trial. Safety will be monitored throughout the trial. Patients completing the trial will be continued on therapy following an extended protocol for as long as safety and efficacy conditions warrant it until BLA approval.

Patient Number and Enrollment Rate

A single patient will be enrolled at the onset of the trial, with two additional patients one month later, and two more patients two weeks later barring any unforeseen complications related to treatment. Additional patients will be admitted should any of the enrolled patients become critically ill, or if a child is in need of an acute clinical procedure for life threatening or harmful conditions.

Diagnosis and Inclusion/Exclusion Criteria

The patient may be male or female, aged five years or older with a documented diagnosis of MPS VI confirmed by measurable clinical signs and symptoms of MPS VI, and supported by a diminished fibroblast or leukocyte ASB enzyme activity level. Female patients of childbearing potential must have a negative pregnancy test (urine β-hCG) just prior to each dosing and must be advised to use a medically accepted method of contraception throughout the study. A patient will be excluded from this study if the patient has previously undergone bone marrow transplantation; is pregnant or lactating; has received an investigational drug within 30 days prior to study enrollment; or has a medical condition, serious intercurrent illness, or other extenuating circumstance that may significantly decrease study compliance.

Dose. Route and Regimen

Patients will receive rhASB at a dose of 1 mg/kg (˜50 U/kg) for the first 3 months of the study. In the event that excess urine GAGs are not decreased by a reasonable amount and no clinical benefit is observed, the dose will be doubled. Dose escalation will occur only after all 5 patients have undergone 3 months of therapy. This rhASB dosage form will be administered intravenously over approximately a four-hour period once weekly for a minimum of 12 consecutive weeks. A peripheral intravenous catheter will be placed in the cephalic or other appropriate vein and an infusion of saline begun at 30 cc/hr. The patient will be premedicated with up to 1.25 mg/kg of diphenylhydramine i.v. based on titration experiments completed prior to the trial. rhASB will be diluted into I 00 cc of normal saline supplemented with 1 mg/ml human albumin. The diluted enzyme will be infused at 1 mg/kg (about 50 units per kg) over a 4 hour period with cardiorespiratory and pulse oximeter monitoring. The patients will be monitored clinically as well as for any adverse reaction to the infusion. If any unusual symptoms are observed, including but not limited to malaise, shortness of breath, hypoxemia, hypotension, tachycardia, nausea, chills, fever, and abdominal pain, the infusion will be stopped immediately. Based on clinical symptoms and signs, an additional dose of diphenylhydramine, oxygen by mask, a bolus of i.v. fluids or other appropriate clinical interventions such as steroid treatment may be administered. If an acute reaction does occur, an assessment for the consumption of complement in the serum will be tested. A second i.v. site will be used for the sampling required for pharmacokinetic analysis.

Evaluable patients

The data from any given patient will be considered evaluable as long as no more than two non-sequential infusions are missed during the 12 weeks of therapy. The initial, midpoint and final evaluations must be completed.

Safety

The enzyme therapy will be determined to be safe if no significant acute reactions occur that cannot be prevented by altering the rate of administration of the enzyme, or acute antihistamine or steroid use. The longer-term administration of the enzyme will be determined to be safe if no significant abnormalities are observed in the clinical examinations, clinical labs, or other appropriate studies. The presence of antibodies or complement activation will not by themselves be considered unsafe, but such antibodies will require monitoring by ELISA, and by clinical assessments of possible immune complex disease.

Efficacy

One purpose of this study is to evaluate potential endpoints for the design of a pivotal trial. Improvements in the surrogate and clinical endpoints are expected as a result of delivery of enzyme and removal of glycosaminoglycan storage from the body. Dose escalation will be performed if mean excess urinary glycosaminoglycan levels are not reduced by a reasonable amount over three months and no significant clinical benefit is observed at 3 months. Improvements are expected to be comparable to those observed in the recently completed MPS I clinical trial and should include improved airway index or resolution of sleep apnea, improved joint mobility, and increased endurance.

Although the invention has been described with reference to the presently preferred embodiments, it should be understood that various modifications can be made without departing—from the spirit of the invention. Accordingly, the invention is limited only by the following claims.

### EXAMPLE 2

A comprehensive review of the available information for the MPS VI cat and relevant pharmacology and toxicology studies is presented below: Enzyme replacement therapy has been established as a promising treatment for a variety of inherited metabolic disorders such as Gaucher Disease, Fabry Disease and Mucopolysaccharidosis I. In some of these disorders a natural animal model offers the ability to predict the clinical efficacy of human treatment during pre-clinical studies. This was found to be true in MPS I (canine model)³². With this in mind, studies have been performed with the MPS VI cat prior to the commencement of human studies for this disease. Sufficient safety and efficacy data exist to proceed with a clinical trial in human MPS VI patients.

Studies of rhASB MPS VI cats indicate that no cat has died as a result of drug administration. As predicted, experiments in MPS VI cats also indicate that rhASB uptake is dependent on the presence of mannose 6-phosphate modified carbohydrate sidechains. RHASB in MPS VI cats has also been shown to clear storage from a variety of major organs and moderately alters bone density. Long-term dose-ranging efficacy studies suggest that a dose of 1 mg/kg/week is the lowest concentration to see significant clinical benefits. Studies has also been performed to compare enzyme distribution, clearance of tissue glycosaminoglycan storage, and decrease of urinary glycosaminoglycan levels after bolus and slow (2 hour) infusion. Studies in progress continue to evaluate the safety of weekly infusions of the projected clinical dose of 1 mg/kg of rhASB in cats suffering from MPS VI.

A spontaneous form of MPS VI in several families of Siamese cats was identified in the 1970's (Jezyk,. Science 198:834-36 (1977)), and detailed reports of the pathological changes in these animals have been published (Haskins, et al., Am J. Pathol. 101:657-674 (1980); Haskins et al.,. J. Am. Vet. Med. Assoc. 182:983-985 (1983); Konde, et al., Vet. Radiol. 28:223-228 (1987)). Although the clinical presentation of these cats is somewhat variable, they all exhibit general changes that have been reported in the literature (Jezyk et al., Science 198:834-36 (1977); Konde et al., Vet. Radiol. 28:223-228 (1987); Crawley, “Enzyme replacement therapy in a feline model of mucopolysaccharidosis type VI” PhD thesis, University of Adelaide, Adelaide, S. Australia, (1998)). Table 6 has been constructed from these sources to provide the “average” changes one would expect to observe in an untreated MPS VI cat:

TABLE 6
MPS VI Cat Model

| | | |
|---|---|---|
| | | Changes Relative to Human |
| Clinical Observation | Timing of Onset | disease (independent of time) |

| | | |
|---|---|---|
| Facial dysmorphia: | 2 months | Similar to human disease |
| Small head, | | |
| Broad maxilla, | | |
| Small ears | | |
| Diffuse corneal clouding | 2 months | Similar to human disease |
| Bone abnormalities: | First signs at 2 | Similar to human disease - |
| Epiphyseal dysplasia, | months - progressive | alterations in enchondral |
| Subluxations, | | calcification |
| Pectus excavatum | | |
| Reduced body weight | 3 months | Similar to human disease |
| Reduced cervical spine | Normal cat value is | Similar to human disease |
| flexibility | 180° at all ages. | |
| | In MPS VI: | |
| | 3 months: 130-170° | |
| | 5 months: 45-130° | |
| | 6 months: 30-100° | |
| | 11 months: 20-80° | |
| Osteoporosis/Degenerative | 1 year or more | Similar to human disease |
| Joint Disease | | |
| Hind limb gait defects | See table below | Carpal tunnel syndrome |
| Hind limb paresis or paralysis | | C₁-C₂ subluxation, |
| (thoracolumbar cord | | Cervical cord compression |
| compressions) | | secondary to thickened dura |
| | | more typical |
| Grossly normal liver and | | Liver and spleen enlarged |
| spleen | | in humans |
| Thickened cardiac valves | | Similar to human disease |
| No CNS lesions - mild lateral | | May be comparable to |
| ventricle enlargement | | hydrocephalus in human |
| | | disease |

Other biochemical/morphological determinations indicate that by 35 days, organs of untreated cats have maximal storage of glycosaminoglycans in tissues (Crawley, A. C. et al. J. Clin.Invest. 99:651-662 (1997)). Urinary glycosaminoglycan levels are elevated at birth in both normal and MPS VI cats but after approximately 40 days, normal cats have decreased levels. MPS VI cats urinary glycosaminoglycans remain elevated or continue to increase until reaching steady state after approximately 5 months.

Variability in clinical presentation is seen in affected littermates. In addition to some variability in the timing of onset of particular abnormalities, the time course of progression for some of the clinical and pathological changes is also variable. In general, the bone lesions are typically progressive (Konde et al., Vet. Radiol. 28:223-228 (1987)), while the corneal clouding is not. In addition, some paralyzed cats have been noted to improve to severe paresis with time. Studies detailing disease progression in individual cats are limited to clinical (or radiographic) observations. Some of these have distinct pathological correlations, such as neurological deficit and cord compression secondary to proliferation of bony tissue in the thoracolumbar region (Haskins et al., J. Am. Vet. Med. Assoc. 182: 983-985 (1983)).

A six-month efficacy study enzyme replacement therapy using recombinant feline ASB in newborn MPS VI cats was conducted. This was prompted by the observation that several treated MPS VI cats developed antibodies to the human enzyme (refer to section 6.5). These antibodies may alter uptake and stability of the enzyme (Brooks et al., Biochim. Biophys. Acta 1361 203-216 (1997)). Feline enzyme was infused at 1 mg/kg weekly. The major conclusions of the study were that urinary GAG, body weight/growth, bone morphometry and clearance of stored material from several tissues was improved relative to the same dose of human recombinant enzyme used in the previous study, that antibodies were not detected beyond the range observed in normal cats, and that the feline enzyme dose at 1 mg/kg was comparable in reversing disease as the human enzyme dose at 5 mg/kg in a head-to-head comparison (Bielicki et al., J. Biol. Chem., in press, 2000). These studies indicate that an incremental improvement in endpoints and immunogenicity is possible when the cat-derived enzyme is given to cats. This provides additional support to dosing human patients with the human enzyme at 1 mg/kg/week. The results of this study are set forth in Table 6.

TABLE 7
Efficacy of Weekly Bolus Injections of CHO-derived Recombinant
Feline ASB in Newborn MPS VI Cats

| | Results | |
|---|---|---|
| | Dose | |
| | 1 mg/kg | |
| | Duration | |
| | 6 months (n = 2) | 3 months (n = 3) |
| Urinary GAGS | Decreased to 2x normal | Decreased to 2x normal |
| Antibody titers | Within range observed in normal cats | To be completed |

Clinical

| | | |
|---|---|---|
| Appearance | Persistent corneal clouding | Persistent corneal clouding |
| | Some resolution of facial dysmorphia | Some resolution of facial dysmorphia; |
| | Improved body shape | Improved body shape |
| Weight | Heavier than normal | Slightly lighter than normal |
| Spine Flexibility | 160°-180° | Not examined |
| (normal = 180°) | | |
| Neurological | Normal | Normal |
| Radiology | Improved quality | Not examined |
| | Density and dimensions of bone | |
| | (similar to 1 mg/kg rh4S in ref. | |
| | 10) | |

Gross

| | | |
|---|---|---|
| Bone/Cartilage | Variable; decreased cartilage | Not examined |
| Thickness | thickness | |
| | more uniform subchondral bone | |
| | (similar to 1 mg/kg rh4S^{a}) | |
| Spinal Cord | No compressions present | Not examined |

Cellular Level

| | | |
|---|---|---|
| Liver (Kupffer) | Complete lysosomal storage | Complete lysosomal |
| | clearing | storage clearing |
| Skin | Almost complete reduction is | Mild reduction |
| | storage | |
| Cornea/Cartilage | No clearance of lysosomal | No clearance of lysosomal |
| (ear, articular) | storage compared with | storage |
| | untreated MPS VI controls | |
| Heart Valves | Significant reduction in | To be completed |
| | lysosomal storage | |
| Aorta | Almost complete reduction in | Mild reduction in |
| | lysosomal storage | lysosomal storage |

Table 8 provides a summary of all studies performed using recombinant human ASB in the MPS VI cat model.

TABLE 8
RhASB Study Results

| No. | | Duration | Route of | | |
|---|---|---|---|---|---|
| Cat | Dose | (Mo.) | Administration | Urinary GAGS | Histopathology |
| 1 | 0/8 mg/kg/14 d | 7-22 | Bolus i.v. | Decreased 50% | Normalization of vacuolization in liver |
| | 1.5 mg/kg/7 d | 22-27 | Bolus i.v. | compared to untreated cat | Significant reduction in kidney and skin |
| | | | | | No correction in cornea and chondrocytes |
| 1 | 0.5 mg/kg/14 d | 12-23 | Bolus i.v. | Decreased to near | No kidney immune complex deposition |
| | 1.4 mg/kg/7 d | 23-27 | Bolus i.v. | normal | |
| 1 | 0.8 mb/kg/14 d | 2-15 | Bolus i.v. | | |
| 1 | 0.2 mg/kg/8 d | 6 | Bolus i.v. | Marginal reduction | N/A |
| | | | | compared to untreated | |
| 4 | 1 mg/kg/7 d | 5/6 | Bolus i.v. | Decreased and | Complete lysosomal storage clearing in liver cells |
| | | | | maintained at 3 × | No evidence or renal impairment or glomerular immune complex |
| | | | | normal compared to | deposition |
| | | | | untreated at 10 × | Significant reduction of lysosomal storage in heart valves |
| | | | | normal | Gradient storage content from media to adventia in aorta |
| 1 | | 11 | Bolus i.v. | | Mild reduction of lysosomal storage of skin (hip joint, dura, kidney) |
| | | | | | No evidence of renal impairment or glomerular deposition |
| | | | | | No significant changes in lysosomal storage of cornea/cartilage |
| 2 | 5 mg/kg/7 d | 5/6 | Bolus i.v. | Decreased and | Complete lysosomal storage in clearing in liver and skin (hip joint, |
| | | | | maintained at 2 × | dura, kidney) |
| | | | | normal compared to | No evidence of renal impairment or glomerular deposition |
| | | | | untreated at 10 × | Near complete reduction in lysosomal storage in heart valves |
| | | | | normal | Thin band of vacuolated cells in outer tuncia media |
| 1 | | 11 | Bolus i.v. | | No evidence of renal impairment or glomerular deposition |
| | | | | | Near complete reduction of lysosomal storage in heart valves |
| | | | | | Thin band of vacuolated cells in outer tuncia media |
| 2 | 0.5 mg/kg | 6 | Bolus i.v. | Decreased to 3 × | Complete lysosomal clearing in liver |
| | 2x weekly | | | normal | Mild to moderate reduction in skin |
| | | | | | Variable reduction of lysosomal storage of heart valves |
| | | | | | Mild reduction of lysosomal storage in aorta |
| 2- | 1 mg/kg/7 d | 1 | Long infusion | Reduced after first or | Reduction of lysosomal storage in reticuloendothelial cells and very |
| | | | (2 hr) | second infusion to | mild in heat valve and aorta after 5 infusion |
| 2 | | | Short infusion | below untreated MPS | |
| | | | (10 min) | VI cats | |
| 5 | 1 mg/kg/7 d | 6 | Long infusion | | |
| | | | (2 hr) | | |

### EXAMPLE 3

### Distribution and Feasibility

An initial study was performed to document enzyme uptake and distribution, and to serve as a pilot study of potential endpoints for future efficacy studies (Crawley et al., J.Clin.Invest. 97:1864-1873 (1996)). Recombinant human ASB was administered by bolus injection to affected cats once per week or once every two weeks at 0.5 up to 1.5 mg/kg. Evaluation of one untreated MPS VI cat (Cat D), and one normal cat provided the values from which comparisons were drawn. The data from the one untreated cat was further supported by historical assessment of 38 additional untreated cats. The acute uptake and distribution studies were conducted in normal cats using an immune assay technique that allowed the detection of human ASB in the presence of normal cat enzyme.

The major conclusions of these studies demonstrated wide uptake of enzyme with the expected predominance of liver and spleen uptake as observed in other enzyme replacement studies in MPS animal models. The uptake efficiency was dependent on the presence of mannose 6-phosphate modified carbohydrate side-chains on the enzyme. The half-life of the enzyme was determined to be 2-4 days. Therapeutically, the enzyme did clear storage from a variety of major organs and did moderately alter bone density. The cornea, bone morphology and cartilage defects were not effectively treated in older MPS VI cats. The study results are summarized in Table 9.

TABLE 9
Summary: Distribution/Feasibility MPS VI Cat Study

| Parameter | Findings | | | | |
|---|---|---|---|---|---|
| Cat | A | | B | | C |
| | Treated MPS VI | | Treated MPS VI | | Treated MPS VI |
| Dose | 0.8 mg/kg | 1.5 mg/kg | 0.5 mg/kg | 1.4 mg/kg | 0.8 mg/kg per 14 d |
| | per 14 d | per 7 d | per 14 d | per 7 d | |
| Age at dose (mo.) | 7*-22 | 22-27 | 12*-33 | 23-27 | 2*-15 |

| Infusion Parameters | 2-10 ml (PBS) via cephalic v. for 5-20 minutes | | |
|---|---|---|---|
| Plasma t_{1/2} (i.v. bolus) | 13.7 ± 3.2 min @ 1 mg/kg | | |
| | 45 min @ 7.5 mg/kg | | |
| | All values relative to endogenous feline ASB enzyme four hours after | | |
| | infusion of 1 mg/kg rhASB in normal cats | | |
| | Liver: 495× | | |
| | Spleen: 6× | | |
| | Lung: 22.3× | | |
| | Heart: 4.3× | | |
| | Aorta: 4× | | |
| | Skin: 31× | | |
| | Cartilage: 0× | | |
| | Cornea: 0× | | |
| Tissue t_{1/2} | 2-4 days @ 1 mg/kg in most organs (detectable enzyme in most tissues | | |
| | of cat B, but only in liver of A after 7 days) | | |
| Neurological | Ambulation | N/A | Marginal progression |
| | fluctuated, but | | to paretic gate by |
| | improved on higher | | end of study |
| | dose | | |
| Corneal Opacity | Did not change with therapy (slit lamp exam 3x late in rx) | | |
| Skeletal (x-rays) | Lesions progressed (no radiographic improvement | | |
| 4 views every 3 mo. | Increased bone volume/trabecular # in cat C (received earlier rx) | | |
| | Vertebral compression in cat C | | |
| Anaphylaxis | No anaphylaxis, minimal distress on infusion; | | |
| Antibody response | 1 × 10⁶ | 64,000 | 64,000 |
| (Ig titers) | (plasma could inhibit | | |
| Untreated MPS VI = | enzyme activity in vitro) | | |
| 4,000-32,000 | | | |

| | | |
|---|---|---|
| Urinary GAGS | Decreased 50% | Decreased to near normal |
| (at ˜ 400 days) | compared to | |
| | untreated cat | |

| | |
|---|---|
| Urinary dermatan | Midway for all 3 cats (relative to untreated control D and normal) |
| sulfate (˜400 days) | |
| Body Weight | 2.5-3.0 kg vs. normal 4-7 kg |
| Liver/Spleen | Grossly normal |
| Heart Valves | Grossly normal |
| Cartilage | Abnormal thickness and formation |
| Microscopy | Normalization of vacuolization in liver, |
| (vacuolization) | Significant reduction in kidney and skin, |
| | No correction in cornea and chondrocytes |
| Kidney immune | Absent |
| complex deposition | |

### EXAMPLE 4

### Efficacy in MPS VI Cats Treated from Birth

A long term dose-ranging efficacy study was performed in MPS VI cats starting at birth (Crawley, et al., J. Clin. Invest. 99:651-662 (1997)), and is summarized in Table 10. MPS VI cats were treated weekly with bolus i.v. injections of 0.2, 1 and 5 mg/kg of rhASB beginning at birth. A total of 9 cats were treated for 5, 6 or 11 months. In addition, 12 MPS VI and 9 normal cats were included as untreated controls. The major conclusions are that 0.2 mg/kg dose did not alter disease progression in the one cat studied, and the only documented clinical benefit was a reduction in the storage in liver Kupffer cells. Urinary GAG levels decreased to near normal during the trial in the higher dose groups. In addition to improvements in the major organs, the higher doses of therapy from birth were able to prevent or ameliorate the bony deformity of the spine and the abnormal form of many bones. There was a dose-dependent effect on improvement in L-5 vertebral bone mineral volume, bone trabecular thickness, and bone surface density between the 1 and 5 mg/kg doses, although both were equivalent in improving bone formation rate at 5 to 6 months of ERT (Byers et al., Bone 21:425-431 (1997)). The mitral valve and aorta was dependent on dose and was less complete at 1 mg/kg but nearly complete at 5 mg/kg. No improvement of storage in cartilage and cornea was observed at any dose. The study suggests that the 1 mg/kg/week dose is the lowest concentration to see significant clinical benefit. The study results are summarized in Table 10.

TABLE 10
Efficacy of Weekly Bolus Injections of CHO-derived Recombinant
Human ASB in Newborn MPS VI Cats (Study PC-BM102-002)^{11, 24}

| | Results | | | |
|---|---|---|---|---|
| | Dose | | | |
| | 1 mg/kg | | 5 mg/kg | |
| Duration | 5/6 mo | 11 mo | 5/6 mo | 11 mo |
| N | 4 | 1 | 2 | 1 |
| Biochemical | | | | |
| Urinary GAGs | Decreased and maintained at 3 × | | Decreased and maintained at 2 × | |
| | normal compared to untreated at 10 × | | normal compared to untreated at 10 × | |
| | normal | | normal | |
| Clinical | | | | |
| Appearance | Variable changes; | | Variable changes; | |
| | Persistent corneal clouding by slit | | Persistent corneal clouding by slit | |
| | lamp | | lamp | |
| Weight | Intermediate (no rx vs. normal) | | Intermediate (no rx vs. normal) | |
| Spine Flexibility | 130-160° | 90° | 180° | 160° |
| (normal = 180) | | | | |
| (untreated MPS | | | | |
| VI = 90°) | | | | |
| Neurological | 1 of 4 mild | No deficits | No deficits | No deficits |
| | hindlimb paralysis | | | |
| Radiology | Improved bone quality, density and | | Improved bone quality, density | |
| | dimensions | | and dimensions | |
| | | | Possibly superior to 1 mg/kg | |
| Gross | | | | |
| Bone/Cartilage | Variability, but | Degenerative | Variability, but | Degenerative |
| Thickness | improved | joint disease | improved | joint disease |
| | | present | | present |
| Spinal Cord | 1 of 4 with | No | No cord compressions | |
| | several mild | compressions | | |
| | compressions | | | |
| Cellular Level | | | | |
| Liver (Kupffer) | Complete | Maintained | Complete | Maintained |
| | lysosomal storage | | lysosomal storage | |
| | clearing | | clearing | |
| Skin (hip Joint, | No evidence of | Mild reduction | Complete | Maintained |
| Dura, Kidney) | renal impairment or | in lysosomal | lysosomal storage | No evidence |
| | glomerular immune | storage | clearing | of renal |
| | complex deposition | No evidence of | No evidence of | impairment or |
| | | renal impairment or | renal impairment or | glomerular |
| | | glomerular | glomerular | deposition |
| | | deposition | deposition | |
| Cornea/Cartilage | NA | No significant | NA | No |
| (ear, articular) | | changes in | | significant |
| | | lysosomal storage | | changes in |
| | | | | lysosomal |
| | | | | storage |

| | | | |
|---|---|---|---|
| Heart Valves | Significant | Significant | Near complete reduction in |
| | (Variable) | (variable) reduction | lysosomal storage |
| | reduction in | in lysosomal | |
| | lysosomal storage | storage near | |
| | | complete | |

| | | |
|---|---|---|
| Aorta | Gradient of storage content from | Thin band of vacuolated cells in |
| | media to adventitia | outer tunica media |

### EXAMPLE 5

### Efficacy of Twice Weekly Infusions of Recombinant Human ASB in Newborn MPS VI Cats

A six-month study was performed in newborn cats to evaluate a 0.5 mg/kg infusion given twice weekly. In addition, the enzyme used in this study was derived exclusively from the CSL-4S-342 cell line. The major conclusions of the study include that compared with the previously reported 1 mg/kg weekly dose, this study produced similar improvements in physical, biochemical, neurological and radiographic parameters. The most notable differences were slightly worsened cervical spine flexibility, and less clearance of lysosomal storage in the denser connective tissues such as the heart valves and aorta. The results are summarized in Table 11.

TABLE 11
Efficacy of Twice Weekly Bolus Injections of CHO-derived Recombinant
Human ASB in Newborn MPS VI Cats

| Parameter | Results |
|---|---|
| Dose | 0.5 mg/kg |
| Duration | 2x weekly: 6 months (n = 2; cats 225 f, 226 m) |
| Urinary GAGs | Decreased to 3 × normal |
| Antibody titres | Within range observed in normal cats |
| Clinical | |
| Appearance | Persistent corneal clouding |
| | Some resolution of facial dysmorphia |
| | Improved body shape |
| Weight | Intermediate (between no treatment and normal) |
| Spine Flexibility | 90°-150° |
| (normal = 180°) | |
| Neurological | No hindlimb paralysis |
| Radiology | Improved quality, density and dimensions of bone (similar to 1 mg/kg |
| | rh4S in ref. 110 |
| Gross | |
| Bone/Cartilage | Variable; decreased cartilage thickness and more uniform subchondral |
| Thickness | bone (similar to 1 mg/kg rh4S^{a}) |
| Spinal Cord | No compressions present |
| Cellular Level | |
| Liver (Kupffer) | Complete lysosomal clearing |
| Skin | Mild to moderate reduction in storage |
| Cornea/Cartilage | No clearance of lysosomal storage compared with untreated MPS VI |
| (ear, articular) | controls |
| Heart Valves | Variable reduction in lysosomal storage (complete in 225 f; no change |
| | from untreated in 226 m) |
| Aorta | Mild reduction in lysosomal storage |

### EXAMPLE 6

### Evaluation of Enzyme Uptake and Distribution as a Function of the Rate of Enzyme Infusion in MPS VI Cats

The primary goal of this study was to compare enzyme distribution, clearance of tissue GAG storage, and decrease of urinary GAG levels after bolus infusion and after slow (2 hour) infusion of an identical 1 mg/kg dose. The slow administration proposal is based on experience from preclinical and clinical studies of α-L-iduronidase for the treatment of MPS I. In addition, the study provided the first data that enzyme produced at BioMarin from cell line CSL-4S-342 is biologically active and safe. Major conclusions of the study include that all four cats (two per group) treated in this study showed no acute adverse reaction to either the slow or fast infusion, and no detrimental effects of repeated enzyme infusions. However, bolus infusion results in high liver uptake which is not preferred. Slow infusion provides better distribution into tissues and therefore is a preferred method for clinical trial.

The tissue distribution of rhASB obtained in the study suggested that 2-hour infusions might increase enzyme levels in other organs apart from the liver, including increased activity in the brain. Reduction in urinary GAG was observed immediately after the first or second infusion to levels below the range observed in untreated MPS VI cats. Correction of lysosomal storage was observed in reticuloendothelial cells and very mild in some fibroblasts (heart valve) and smooth muscle cells (aorta) after 5 infusions. No other significant clinical response to infusions was observed in either group, however this was not unexpected due to the short duration of the study, and due to therapy starting after significant disease changes had already developed. The extended 2-hour infusion was safe and well tolerated relative to the shorter protocols used in previous studies. The 2-hour infusion may provide improvement in enzyme distribution based on the one cat that was evaluable for enzyme tissue distribution.

### EXAMPLE 7

### Month Safety Evaluation of Recombinant Human N-acetylgalactosamine-4-sulfatase in MPS VI Affected Cats

Two 6 month studies in MPS VI cats have been initiated using the enzyme produced by the manufacturing process according to the present invention. The purpose of these studies is to evaluate the safety and efficacy of weekly infusions of the projected human clinical dose of rhASB in cats suffering from MPS VI. Study 6 involves kittens dosed initially at 3 to 5 months of age. Study 7 involves kittens treated from birth with weekly infusions of the projected human clinical dose of rhASB. The studies are intended to access potential toxicology. Cats will be observed for changes in behavior during infusion of the recombinant enzyme to assess possible immune responses. Serum will be monitored for complement depletion and for the formation of antibody directed against the recombinant enzyme. General organ function will be monitored by complete clinical chemistry panels (kidney and liver function), urinalysis, and complete blood counts (CBC) with differential. Urinary glycosaminoglycan levels will be monitored on a weekly basis at a set time points relative to enzyme infusion. Evidence of clinical improvements in disease will be documented. These data will provide additional assessment of the potential efficacy of the treatment and will validate the activity and uptake of the enzyme in vivo. The studies have and will be conducted in a manner consistent with the principles and practices of GLP regulations as much as possible.

Preliminary results of the first study indicate that administration of rhASB has not had any detrimental effects on any of the animals, with bodyweights and clinical chemistries generally maintained within reference ranges. However, both of the cats with significantly elevated antibody titers developed abnormal clinical signs during infusions, however both animals behaved normally once enzyme infusions ceased and did not appear to suffer any longer tem ill effects. Extended infusion times (4 hours) and increased premedication antihistamines have allowed continued therapy in the cats without any abnormal clinical signs. Mild reduction in urinary GAG levels suggest some efficacy of therapy in reducing stored glycosaminoglycans in tissues or circulation, however fluctuations in these levels were observed over time making interpretation difficult. None of the 5 cats have shown obvious clinical improvements in response to ERT, but this will require at least 6 month treatment based on previous studies²³. Antibody titers have developed in four out of the five cats, with noticeable increases in titers observed after 2 months of ERT. Two of these cats have developed significantly elevated titers after 2 or 3 months.

### EXAMPLE 8

### Safety Profile for MPS VI Cats Treated with rhASB

A study has commenced enrolling affected cats that were treated within 24 hours of birth. Forty-one MPS VI cats have been treated using rhASB. Administration of enzyme to normal cats has been restricted to one to two cats to confirm acute safety of new batches prior to exposure of the valuable affected animals to therapy. In summary, no MPS VI cat has died as a result of drug administration, although four cats have died as a result of viral infection or an underlying congenital abnormality. Enzyme for the studies was produced according to the production methods of the present invention. The preliminary data are set forth in Table 12.

TABLE 12
MPS VI Cat Efficacy Study Summary from Hopwood Laboratory

| | | Dose/wk | Rx Length | |
|---|---|---|---|---|
| Study #¹ | # of Cats | (mg/kg) | (mos.) | Mortality |
| 1 | 2 | Variable | 13-21 | None |
| | 1 | | | |
| 2 | 1 | 0.2 | 5 | None |
| 2 | 1 | 0.2 | 1 | Died: congenital |
| | | | | heart defect |
| — | 2 | 0.5 | 3-5 | 1 died parvovirus |
| 2 | 4 | 1 | 3-11 | 1 died parvovirus |
| | 2 | | | |
| — | 1 | 1 | 6 | None |
| | | | (s.c.) | |
| — | 4 | 1 | 6 | None |
| 2 | 2 | 5 | 3-11 | 1 died parvovirus |
| | 2 | | | |
| 4 | 2 | 0.5 | 5 | None |
| | | (twice) | | |
| — | 3 | 0.5 | 5 | None |
| | | (twice) | | |
| 5 | 4 | 1 | 1 | None |
| 6 | 5 | 1 | Started | None |
| | | | Jul. 21, 1999 | |
| 7 | 5 | 1 | Started | None |

Although the invention has been described with reference to the presently preferred embodiments, it should be understood that various modifications can be made without departing—from the spirit of the invention. Accordingly, the invention is limited only by the following claims.

1-32. (canceled)

33. A pharmaceutical composition comprising recombinant N-acetylgalactosamine-4-sulfatase, a pharmaceutically acceptable carrier, and a polyoxyethylenesorbitan.

34. A pharmaceutical composition comprising recombinant N-acetylgalactosamine-4-sulfatase, a pharmaceutically acceptable carrier, and polyoxyethylenesorbitan 20 or 80.

35. A pharmaceutical composition comprising recombinant N-acetylgalactosamine-4-sulfatase, a pharmaceutically acceptable carrier, and polyoxyethylenesorbitan 20.

36. A pharmaceutical composition comprising recombinant N-acetylgalactosamine-4-sulfatase, a pharmaceutically acceptable carrier, and polyoxyethylenesorbitan 80.

37. The pharmaceutical composition of any one of claims 33 through 36, wherein said polyoxyethylenesorbitan concentration is about 0.001% (W/V).

38. The pharmaceutical composition of any one of claims 33 through 36 wherein said recombinant N-acetylgalactosamine-4-sulfatase is a human N-acetylgalactosamine-4-sulfatase.

39. The pharmaceutical composition of claim 37 wherein said recombinant N-acetylgalactosamine-4-sulfatase is a human N-acetylgalactosamine-4-sulfatase.

40. A pharmaceutical composition suitable for administration to humans comprising recombinant human N-acetylgalactosamine-4-sulfatase and a pharmaceutically acceptable carrier, said composition having the following characteristics:
(a) activity by fluorescence assay is between about 20,000 and 120,000 mUnits;
(b) absence of detectable adventitious viruses;
(c) absence of detectable mycoplasma;
(d) endotoxin level by LAL assay of about 2 EU/mL or less;
(e) about 600 or less particulates of 25 um in size per vial;
(f) about 6000 or less particulates of 10 um in size per vial;
(g) pH between about 5.5 and 6.8;
(h) protein concentration of about 0.8 to 1.2 mg/mL;
(i) one major band between about 65 and 70 kDa detectable on SDS-PAGE;
(j) greater than about 95% purity by RP-HPLC; and
(k) calculated specific activity of between about 40,000 and 80,000 mUnits per milligram of protein.

41. The pharmaceutical composition of claim 40 produced by a purification process comprising the steps of:
(a) harvesting fluid obtained from a culture of cells transformed with a gene encoding a recombinant N-acetylgalactosamine-4-sulfatase;
(b) running the fluid on a DEAE sepharose column;
(c) running the fluid on a Blue sepharose FF column;
(d) running the fluid on a copper chelating sepharose column;
(e) running the fluid on a phenyl sepharose column; and
(f) diafiltering the purified recombinant human N-acetylgalactosamine-4-sulfatase.
